(19) United States
(12) Patent Application Publication
(10) Pub. No.: US 2025/0025130 A1
(43) Pub. Date: Jan. 23, 2025

(54) ACCESSORY HOLDER AND SYSTEM FOR ULTRASOUND PROBE

(71) Applicant: SUPERSONIC IMAGINE, Aix-En-Provence (FR)

(72) Inventors: Damien MAJOREL, Aix-en-Provence (FR); Fabien ALBRAND, La Roche-de-Rame (FR)

(21) Appl. No.: 18/760,732

(22) Filed: Jul. 1, 2024

(30) Foreign Application Priority Data
Jul. 21, 2023 (FR) 2307846

(51) Int. Cl. A61B 8/00

(52) U.S. Cl. CPC A61B 8/4444

(57) ABSTRACT

Example embodiments relate to an accessory holder for an ultrasound probe, the accessory holder including a first attachment interface configured to mechanically pair with an ultrasound probe, and a second attachment interface, of a shape complementary to a third attachment interface with which a probe accessory is provided, configured to mechanically pair in a removable manner with said third attachment interface rotating the probe accessory relative to the accessory holder. The invention also relates to an accessory system that includes such an accessory holder and such a probe accessory.

### PRIOR ART

The present invention relates to accessory holders for ultrasound probes and in particular aims at holders and systems intended to attach an accessory to an ultrasonic probe.

Ultrasound devices currently apply to many applications, in the fields particularly of acoustics, material analysis, medical imaging and biomedicine.

For this purpose, an ultrasound device generally comprises an ultrasound probe controlled by means of electrical signals, these signals being for example transmitted between the probe and a control unit. The ultrasound probe (also called ultrasound transducer device) comprises at least one or a set of ultrasound transducer elements intended to transmit and/or receive ultrasound waves to and/or from a medium of interest. Thus, electrical signals representing ultrasound waves can be transmitted to and/or received from these transducer elements, causing the transmission and/or the reception of ultrasound waves into and/or from the medium considered.

More particularly, in a conventional ultrasound imaging method, it is possible to use for example an ultrasonic probe equipped with one or more transducers to convert the electrical signals into ultrasound waves. The transducers may transmit one or successively more ultrasound beams in the direction of a medium, which corresponds to a transmission operation. Subsequently, in a receiving operation, a set of backscattered echo signals are received from the medium by the same set or by another set of transducer elements. In particular, each of the transducer elements can for example convert an echo signal received into an electrical signal. The signal can subsequently be processed by the ultrasound system or by any directly connected or not associated system.

In some use cases, it may be useful to install an accessory on the ultrasound probe. By way of example, it is known to attach a position sensor on an ultrasound probe to enable an imaging system to detect the position of the probe in order to help the user to position the probe relative to the medium of interest. Thus, it is possible to superimpose an image (for example an ultrasound image or an MRI (magnetic resonance imaging) image with a representation of the probe or of its position in order to mark the position of the probe in the medium considered. Thanks to this type of sensor, it is easily possible to identify a position given to the image, for the purposes for example of observation, surgical intervention, biopsy or other.

The correct positioning of such a sensor relative to the ultrasound probe is critical for making it possible to accurately locate the probe in space. Any unexpected movement of the sensor on the probe may in particular result in the detection by the system of a parasitic motion of the probe that does not reflect the real behaviour of the probe.

To prevent the detection of such parasitic motions, the sensor should therefore be attached reliably and robustly to the ultrasound probe. Nevertheless, the installation of such a sensor must not be carried out to the detriment of other constraints, particularly in terms of ergonomics, ease of installation and cleaning and cost.

In order to overcome any problem of parasitic motion, it is possible to directly integrate a position sensor into an ultrasound probe. However, the user of the probe may not always need this sensor, which moreover produces an increase of the weight of the probe and potentially reduces its ergonomics, or its autonomy. The presence of the sensor in the probe may particularly require modifying the external shape of the probe, and/or increasing the diameter of the cable connecting the probe to the control unit which may lead to an increased rigidity of the assembly. Furthermore, it may be desirable to change the sensor according to the use that is made of the ultrasound probe or to use the same sensor on different probes.

It is known to use a sensor holder that makes it possible to attach in a removable manner a position sensor to an ultrasound probe. However, such a holder does not make it possible to offer a removable, reliable and robust attachment of the sensor on the probe, while guaranteeing good ergonomics of use and this at a limited cost. In the event of clipping the sensor on the holder for example, there is a risk that the sensor moves or unexpectedly separates from the probe. If on the contrary attaching by clipping requires excessive force for attaching or detaching the sensor of the probe, this may cause handling problems for the user, particularly if the gripping surface of the sensor is limited or if the user wears gloves. In general, the user needs to be able to rapidly and effectively position or remove the sensor in order to limit the risks of damaging the equipment, optimise the operating time of the machine and avoid unnecessary manipulations. Furthermore, it is generally difficult to rapidly and reliably clean such a sensor holder due to its not very suitable design, which may be problematic in particular in environments where the requirements in terms of hygiene are high, such as particularly in the medical field.

### DESCRIPTION OF THE INVENTION

One of the objects of the present invention is to address at least one of the problems or shortcomings described above.

In particular, one object of the present invention is to offer an accessory holder for ensuring a removable attachment of an accessory to an ultrasound probe, such that this attachment is reliable and robust while guaranteeing good ergonomics and ease of use.

For this purpose, according to a first aspect, the aim of the present invention is an accessory holder for ultrasound probe comprising:
a first attachment interface configured to mechanically pair with an ultrasound probe; and
a second attachment interface, of shape complementary to a third attachment interface with which a probe accessory is provided, configured to mechanically pair in a removable manner with said third attachment interface by rotating the probe accessory relative to the accessory holder.

The accessory holder according to the invention can include other features that can be taken separately or in combination, particularly among the following embodiments that are set out for illustrative purposes only and can be combined or associated unless stated otherwise.

According to one example, one of the second and third attachment interfaces constitutes a male element and the other constitutes a female element, said male and female elements being configured to rotatably mechanically pair by inserting the male element into the female element.

According to one example, the second attachment interface of the accessory holder constitutes the female element.

According to one example, the second attachment interface is configured to mechanically pair in a removable manner with said third attachment interface by mechanical pairing successively comprising inserting the male element into the female element in a direction of insertion and rotating the probe accessory relative to the accessory holder about an axis of rotation corresponding to the direction of insertion.

According to one example, the female element comprises:
at least one insertion slot configured in order to insert therein a respective locking member with which the male element is provided; and
a positioning slide respectively leading to each insertion slot to make it possible to move, by rotating the probe accessory relative to the accessory holder, the respective locking member in the positioning slide up to a locking position thus causing the mechanical pairing of the accessory holder with the probe accessory.

According to one example, one of the second and third attachment interfaces comprises at least one deformable wing configured to deform in response to the movement of the respective locking member in the locking slide up to the locking position so as to cause a frictional force opposing said movement.

According to one example, said at least one deformable wing is comprised in the second attachment interface of the accessory holder.

According to one example, the second attachment interface comprises two deformable wings.

According to one example, one of the second and third attachment interfaces comprises at least one first cavity, said at least one deformable wing and said at least one first cavity being configured so that each deformable wing engages in a first cavity when said at least one locking member reaches the locking position.

According to one example, the second attachment interface comprises at least two insertion slots of different size configured for engagement therein by respective locking members of the third attachment interface.

According to one example, one of the second and third attachment interfaces comprises a second cavity and the other comprises a protuberance configured to engage in the second cavity when said at least one locking member reaches the locking position.

According to one example, the second and third attachment interfaces are configured to be mechanically paired in a removable manner by a ¼ turn rotation of the accessory holder relative to the probe accessory.

According to a second aspect, the aim of the present invention is an accessory holder for ultrasound probe, this accessory holder comprising:
an accessory holder such as defined according to the first aspect of the present invention; and
a probe accessory comprising a third attachment interface of shape complementary to the second attachment interface of the accessory holder, wherein the second and third attachment interfaces are configured to mechanically pair in a removable manner by rotating the probe accessory relative to the accessory holder.

The accessory system according to the invention can include other features that can be taken separately or in combination, particularly among the following embodiments that are set out for illustrative purposes only and can be combined or associated unless stated otherwise.

According to one example, one of the second and third attachment interfaces constitutes a male element and the other constitutes a female element, said male and female elements being configured to rotatably mechanically pair by inserting the male element into the female element.

According to one example, the second and third attachment interfaces are configured to mechanically pair in a removable manner by mechanical pairing successively comprising inserting the male element into the female element in a direction of insertion and rotating the probe accessory relative to the accessory holder about an axis of rotation corresponding to the direction of insertion.

According to one example, the probe accessory comprises at least one of the following:
a sensor;
a gel distributor; and
a light source.

According to one example, the probe accessory comprises a position sensor.

According to one example, the probe accessory forms a rotary knob.

According to a third aspect, the present invention relates to a method for attaching the accessory system such as defined in the second aspect of the present invention. This attachment method comprises:
inserting a locking member of the male element respectively into an insertion slot of the female element; and
moving, by rotating the probe accessory relative to the accessory holder, the locking member in, respectively, the positioning slide up to a locking position thus causing the mechanical pairing of the accessory holder with the probe accessory.

The present invention thus advantageously makes it possible to offer an accessory holder for ultrasound probe ensuring a removable attachment of an accessory to an ultrasound probe, this attachment being reliable and robust while guaranteeing good ergonomics of use and easy, effective and fast cleaning.

The features and advantages of the invention will become apparent more clearly upon reading the description below, provided purely by way of non-limiting example, and with reference to the appended figures. In particular, the examples illustrated in the figures can be combined together, provided that there are no mentioned or evident inconsistencies.

### BRIEF DESCRIPTION OF THE FIGURES

Other features and advantages of the present invention will become apparent from the description of the non-limiting examples of embodiments of the present invention below, with reference to the appended FIGS. 1 to 15, wherein:

FIG. 1 schematically illustrates an ultrasound system comprising an ultrasound probe, according to a particular example;

FIG. 2 schematically illustrates an accessory holder and an accessory system for an ultrasonic probe, according to one example of embodiment of the present invention;

FIG. 3 schematically illustrates the accessory holder and the accessory system of FIG. 2, according to one example of embodiment of the present invention;

FIG. 4 schematically illustrates an accessory holder and an accessory system, according to one example of embodiment of the present invention;

FIG. 5 is an exploded perspective view schematically representing an accessory system comprising an accessory holder and an accessory for ultrasonic probe, according to one example of embodiment of the present invention;

FIG. 6 schematically illustrates a mechanical pairing of a probe accessory with an accessory holder (position P1), according to one example of embodiment of the present invention;

FIG. 7 schematically illustrates a mechanical pairing of a probe accessory with an accessory holder (position P2), according to one example of embodiment of the present invention;

FIG. 8 schematically represents a method for attaching an accessory system for ultrasonic probe, according to one example of embodiment of the present invention;

FIG. 9 schematically illustrates an attachment interface of an accessory holder for ultrasonic probe, according to one example of embodiment of the present invention;

FIG. 10 is a sectional view schematically representing the attachment interface of the accessory holder of FIG. 9, according to one example of embodiment of the present invention;

FIG. 11 is a perspective view schematically representing the attachment interface of the accessory holder according to FIGS. 9 and 10, according to one example of embodiment of the present invention;

FIG. 12 schematically illustrates an attachment interface of a probe accessory, according to one example of embodiment of the present invention;

FIG. 13 schematically illustrates perspective and bottom views of the attachment interface of the probe accessory of FIG. 12, according to one example of embodiment of the present invention;

FIG. 14 schematically illustrates an accessory holder and an accessory system for an ultrasonic probe, according to one example of embodiment of the present invention; and

FIG. 15 is a view representing an ultrasound probe equipped with an accessory holder and a probe accessory, according to one example of embodiment of the present invention.

### DESCRIPTION OF THE EMBODIMENTS

An ultrasound probe, also known as ultrasonic probe or ultrasound device or transducer device, is an ultrasound wave transmitter and/or receiver device, controllable by means of electrical signals, these signals being for example transmitted between the ultrasound probe and a control unit of the control system.

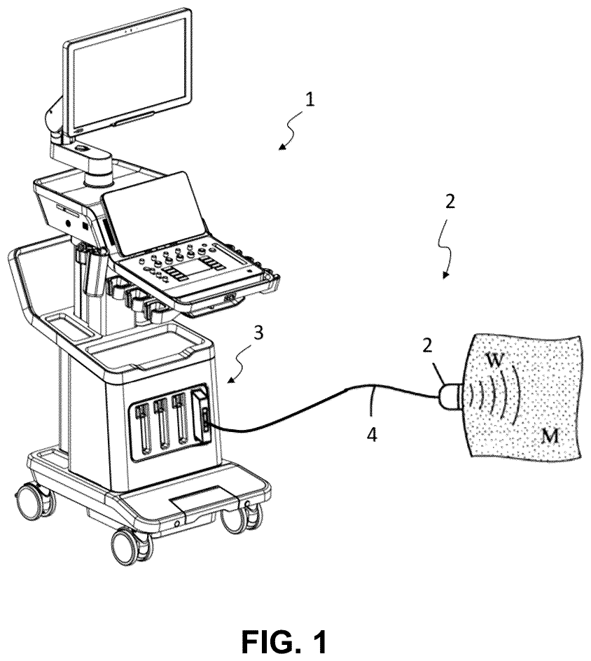

FIG. 1 schematically represents, purely for illustrative purposes, one example of embodiment of an ultrasound system 1 comprising an ultrasound probe 2. This probe 2 may comprise one or more transducer elements (for example one or more piezoelectric elements) configured to transmit and/or receive ultrasound waves W. Each transducer element is thus configured to convert an electrical signal received from the system 1 into ultrasound waves and/or vice versa. The nature of these waves depends on the system 1 and/or on the probe 2, particularly in view of the use that is made thereof and of the objectives to be achieved.

Thus, the transducer elements may be configured to transmit (a) ultrasound waves W (or pulses) into the medium M and/or to receive (b) a plurality of ultrasonic signals W from the medium M, possibly in response to the transmission (a) of waves W.

According to one example, the ultrasound probe 2 is configured to transmit ultrasound waves W. According to another example, the ultrasound probe 2 is configured to receive ultrasound waves W. According to another example, the ultrasound probe 2 is configured to both transmit and receive ultrasound waves W.

The transducer elements of the probe 2 may comprise piezoelectric crystals and/or other components that may be configured to generate and/or record and/or receive signals.

As illustrated, the ultrasound system 1 may comprise a processing unit (or device) 3 configured to control the ultrasound probe 2 by means of electrical signals that are exchanged (or transmitted) between the system 1 and the ultrasonic probe 2.

The processing unit 3 is configured to generate electrical signals that are sent to the ultrasound probe 2 to cause the transmission, by said probe, of ultrasound waves W in the direction and/or into a medium M. The electrical signals thus generated are representative of (or define) the ultrasound waves transmitted into the medium M.

The processing unit 3 may also be configured to process (or interpret) electrical signals received from the ultrasound probe 2. These signals are representative of ultrasound waves received by the probe 2 from the medium M. These waves form for example one or more ultrasounds, that is to say a response from the medium M to the ultrasound waves previously transmitted.

The system 1 may comprise the ultrasound probe 2. Alternatively, the ultrasound probe 2 may be external to the system 1. For example, the ultrasound probe 2 may be connected to the system 1 by a cable 4 (FIG. 1) or may communicate with it wirelessly.

The system 1 may be an imaging system, for example in the medical field. The images generated by the system 1 may either be analysed in real time, for example by a user, or analysed later and/or at a location other than the one where the system 1 is located.

The system 1 may be a medical system, for example an ultrasound system. Consequently, the ultrasound probe 2 is for example an ultrasound probe.

For example, the system 1 may be associated with an ultrasonic probe 2, in order to study a medium M, particularly to collect ultrasound data from such a medium M. The medium M thus observed can take various forms depending on the case. It may be, for example, living tissue and/or in particular human tissue. It is also possible to observe a medium M comprising one or more mineral structures, for example (gravel, volcano, etc.).

According to other examples, the system 1 and/or the ultrasound probe 2 are configured for communication, imaging or scanning purposes, for example in the medical imaging, radar, sonar, seismology, wireless communications, radio astronomy, acoustic and biomedicine field.

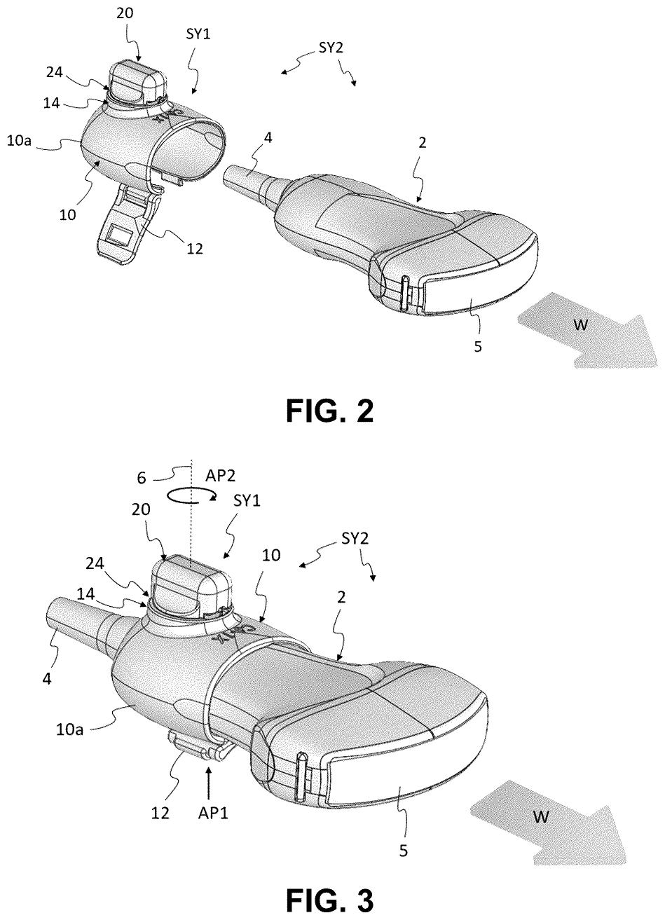

Examples of implementation of an accessory holder 10 for ultrasound probe, and of an accessory system SY1 comprising such a holder 10, as well as a corresponding attachment method, will now be described in the following with joint reference to FIGS. 2-15 provided for illustrative purposes.

Unless stated otherwise, common or similar elements in several figures bear the same reference numerals and have identical or similar features such that these common elements are not generally described again for the sake of simplicity.

The terms “first” (or “first, second”, etc.) are used in this document by arbitrary convention to enable different elements (such as operations, devices, etc.) implemented in the embodiments described below to be identified or distinguished.

In the following example, it is considered purely for illustrative purposes an accessory holder 10, and more generally an accessory system SY1, configured to be attached to an ultrasound probe 2 such as described above with reference to FIG. 1. As can be seen in FIGS. 2-15, the configuration of the accessory holder 10 and of the accessory system SY1, particularly in terms of shape, size, etc., can be adapted according to the case, and particularly depending on the ultrasound probe 2 considered and/or on the accessory 20.

The accessory holder 10 is a holder for ultrasound probe, namely for the ultrasound probe 2 in the examples considered, this holder being configured to hold or attach, in a removable manner, an accessory 20 (also known as probe accessory) to the ultrasound probe 2. The nature and the configuration of this accessory 20 may vary according to the case. Purely for illustrative purposes, it is considered in the following examples that the probe accessory 20 is a second holder, known as device holder, configured to contain or hold an accessory device, namely a sensor for example. Alternative embodiments of this accessory 20 are described hereinafter.

The accessory system SY1 (FIGS. 2-3) comprises the accessory holder 10 and the accessory 20, these two elements 10 and 20 may be attached together in a removable manner as described below. The accessory 20 is a distinct element of the accessory holder 10.

The accessory system SY1 and the ultrasound probe 2 form a probe system SY2 (FIGS. 2-3) in this example.

More precisely, as illustrated in FIGS. 2-4, the accessory holder 10 comprises two attachment interfaces (known as locking interfaces, or attachment devices), namely a first attachment interface 12 and a second attachment interface 14.

The first attachment interface 12 is configured to mechanically pair (or be paired) with the ultrasound probe 2. In other words, this attachment interface 12 is designed to mechanically couple (or attach) together the ultrasound probe 2 and the accessory holder 10. The accessory holder 20 is therefore an element external to (or distinct from) the ultrasound probe 2. The attachment interface 12 may comprise any attachment mechanism for anchoring the accessory holder 10 securely to the probe 2.

In the examples described, the attachment interface 12 is configured to mechanically pair (or associate or attach) in a removable manner the accessory holder 10, and more generally the accessory system SY1, with the ultrasound probe 2. The mechanical pairing (or coupling) of the accessory holder 10 with the probe 2 is noted AP1 (FIG. 3). Once the pairing AP1 has been performed, a user can therefore if necessary detach the accessory holder 10 from the ultrasound probe 2 by a decoupling (or detachment) operation reverse to the pairing operation AP1. Alternative embodiments wherein the mechanical pairing AP1 of the accessory holder 10 on the probe 2 is permanent are nevertheless possible.

As illustrated in FIGS. 2-4, the accessory holder 10 may be configured in order to insert therein the ultrasound probe 2. To this end, the accessory holder 10 may comprise an inner surface of shape complementary to the shape of at least one portion of the ultrasound probe 2. The accessory holder 10 forms for example a cavity configured so that all or part of the ultrasound probe 2 can be inserted therein.

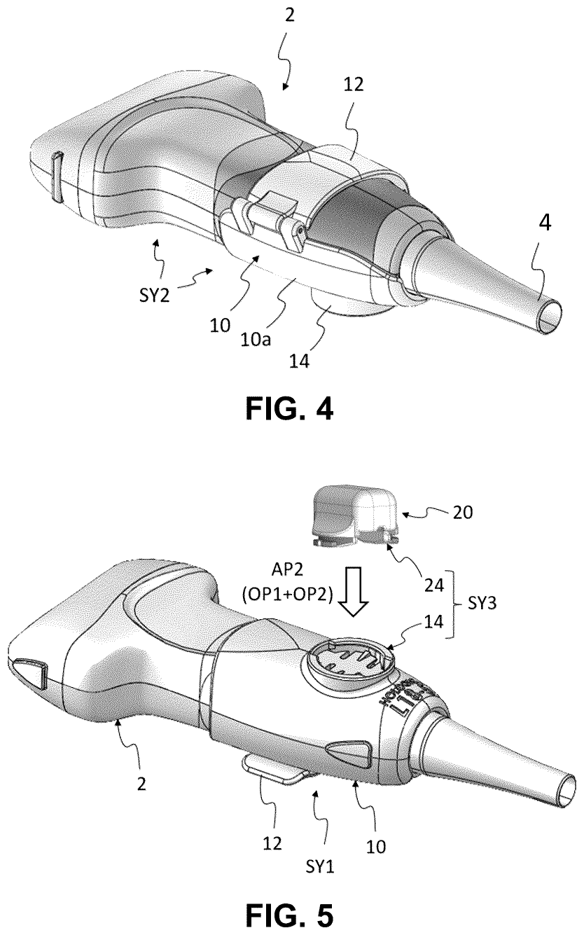

According to one example, the first attachment interface 12 comprises an articulated arm that is rotatably mounted on the body 10a of the accessory holder 10. Once the ultrasound probe 2 has been inserted into the accessory holder 10, the articulated arm may be rotatably moved from an open position into a closed position (and vice versa), thus causing the mechanical pairing AP1 of the accessory holder 10 on the ultrasound probe 2 (and vice versa the mechanical decoupling of these elements). The configuration of this arm may vary according to the case and according to the type of probe used. One example of implementation of this articulated arm is represented in FIG. 4.

The second attachment interface 14 of the accessory holder 10 is of shape complementary to a third attachment interface 24 (FIGS. 2-3) with which the probe accessory 20 is equipped (or provided). In other words, the accessory 20 comprises the third attachment interface 24 of shape complementary to that of the second attachment interface 14. The attachment interface 14 of the accessory holder 10 is thus configured to mechanically pair in a removable manner (or reversibly) with the third attachment interface 24 by rotating the probe accessory 20 relative to the accessory holder 10. The mechanical pairing (or coupling) of the accessory holder 10 with the accessory 20 is noted AP2 (FIG. 3).

The second and third attachment interfaces 14 and 24 together form an attachment system SY3 (FIG. 5).

Their mutual complementarity of shape makes it possible for the attachment interfaces 14 and 24 to mechanically cooperate together to make it possible to attach, in a removable (or reversible) manner, the accessory 20 to the accessory holder 10. Once the pairing AP2 has been performed, a user may therefore if necessary detach (or decouple, or separate) the accessory 20 relative to the accessory holder 10 by a decoupling (or detachment) operation reverse to the pairing operation AP2. The reversible (or removable) character of the pairing AP2 means that a user can selectively attach and detach the attachment interfaces 14 and 24 relative to one another.

The complementarity of shape between the attachment interfaces 14 and 24 can be ensured in various ways. According to one example, one of the second and third attachment interfaces 14 and 24 constitutes a male element and the other constitutes a female element, these male and female elements being configured to rotatably mechanically pair (couple) by inserting the male element into the female element.

As illustrated particularly in FIGS. 2-5, it is assumed hereinafter purely for illustrative purposes that the third attachment interface 24 constitutes the male element and that the second attachment interface 14 constitutes the female element. Thus, the attachment interfaces 14 and 24 are configured to rotatably mechanically pair by inserting (all or part) of the attachment interface 24 into (all or part) of the attachment interface 14.

Alternative embodiments according to the reverse configuration are nevertheless possible. Thus, the examples of embodiments described in the present invention can be applied in a similar way in the alternative case where the second attachment interface 14 constitutes the male element and the third attachment interface 24 constitutes the female element.

The configuration of the second attachment interface 14 as the female element advantageously makes it possible to obtain a mechanical pairing AP2 that is more robust than in the reverse configuration, in particular if the ultrasound probe 2 is considered as a fixed part and the accessory 20 as a movable part. The accessory 20 can for example be qualified as movable part if this part is manually grasped and moved by the user during operations OP1 and OP2 whereas the accessory holder 20 remains substantially immobile in space.

It should be noted that the relative rotation OP1 of the accessory 20 relative to the accessory holder 10 can be performed in various ways, for example by carrying out a rotation of the accessory 20 while maintaining at a fixed position the accessory holder 10, or by carrying out a rotation of the accessory holder 10 while maintaining at a fixed position the accessory 20, or also by carrying out rotations (in the opposite direction) both of the accessory holder 10 and of the accessory 20.

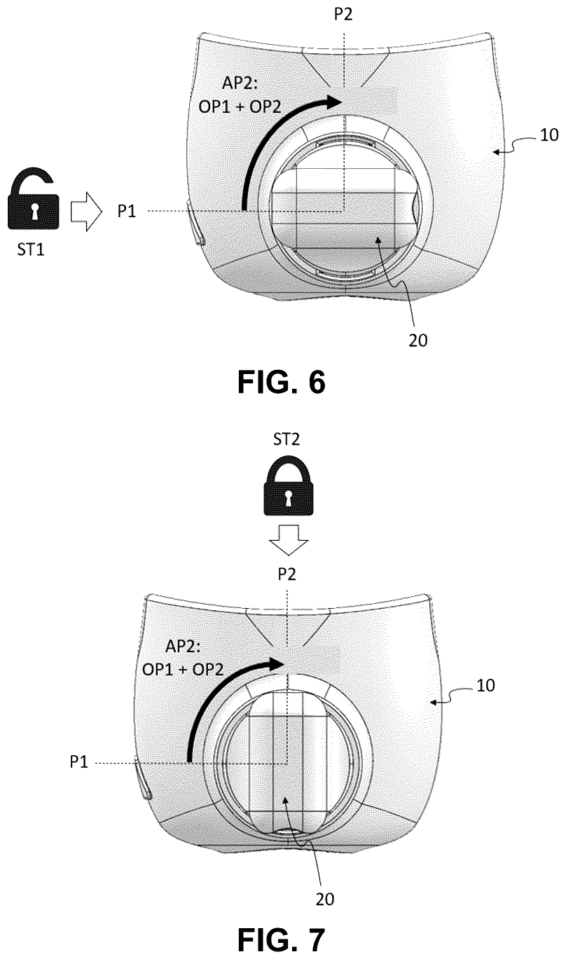

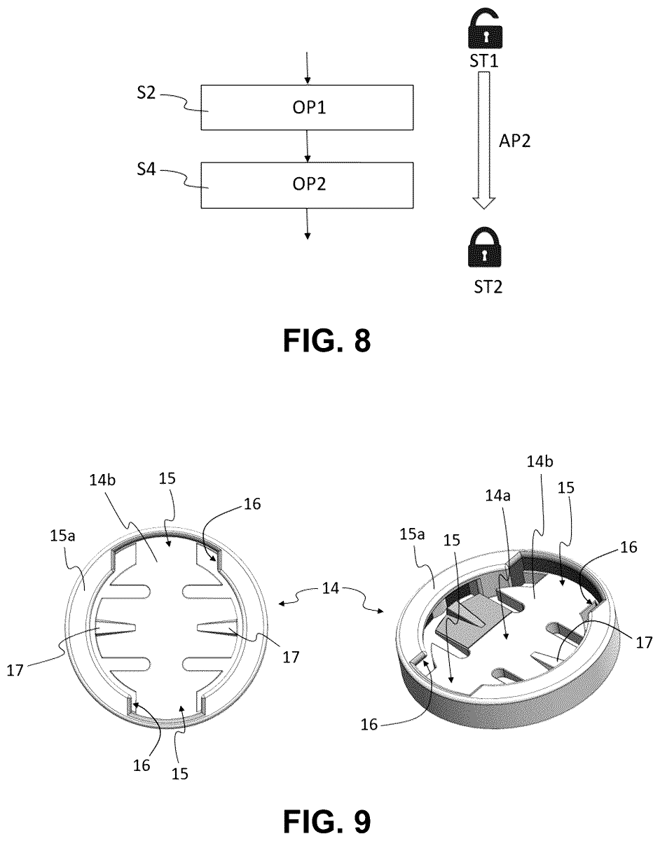

FIGS. 6-7 represent the process for mechanically pairing AP2 the accessory 20 with the accessory holder 10 according to one particular example. FIG. 8 represents in the form of a diagram the steps of an attachment method that aims to attach together the accessory holder 10 and the accessory 20 according to the mechanical pairing AP2.

As illustrated by way of example in FIGS. 3-8, the mechanical pairing AP2 is accomplished by successively performing an insertion operation OP1 and a rotation operation OP2. It is assumed at an initial stage that the attachment interfaces 12 and 24 are in a first unpaired state ST1. During the insertion operation OP1 (Step S2, FIG. 8), the attachment interface 24 of the accessory 20 is inserted (or engaged) OP1 into the attachment interface 14 of the accessory holder 10 (for example by a translational motion). Once the attachment interface 24 has been inserted in position into the attachment interface 14, a rotation OP2 of the accessory 20 relative to the accessory holder 10 is carried out about an axis of rotation AX1 (Step S4, FIG. 8) from a first angular position P1 into a second angular position P2 known as locking position, thus causing the mechanical pairing AP2 of the accessory 20 with the accessory holder 10. The mechanical pairing AP2 thus makes it possible to change the attachment interfaces 14 and 24 from the unpaired state ST1 to a second paired state ST2 (position P2).

The ways in which the insertion OP1 and rotation OP2 operations (or motions) are performed may vary according to the configurations of the attachment interfaces 12 and 14. According to one example particularly illustrated in FIGS. 3 and 5-8, the second attachment interface 14 is configured to mechanically pair in a removable manner with the third attachment interface 24 by mechanical pairing AP2 successively comprising inserting OP1 the male element (namely the third attachment interface 24 in this example) into the female element (namely the second attachment interface 14 in this example) in a direction of insertion 6 and rotating OP2 the probe accessory 20 relative to the accessory holder 10 about an axis of rotation corresponding to the direction of insertion 6. In other words, once the male element has been inserted (or engaged) into the female element by an insertion motion OP1 (by translation) along the axis 6, a rotation OP2 of the probe accessory 20 relative to the accessory holder 10 is carried out about the direction (or the axis) 6 serving as rotational axis. The pairing AP2 is thus performed by successive execution of the operations OP1 and OP2. In this way, advantageously it is possible to mechanically couple in a removable manner the second and third attachment interfaces 14 and 24 by a continuous motion in the same direction 6, namely a translation along the axis 6 then a rotation about this same axis 6. This correspondence of direction between insertion movement OP1 and rotational movement OP2 makes it possible for example advantageously to easily and rapidly pair the accessory holder 10 with the probe accessory 20 by a continuous manual movement successively comprising inserting OP1 then rotating OP2, and this with accuracy and a minimum of manipulation.

Furthermore, the coupling according to the present invention makes it possible to effectively hold the probe accessory, regardless of its weight, including in cases where its weight is greater, or even substantially greater, than that of the probe. Indeed, in particular cases where the probe accessory is relatively heavy relative to the probe, a risk may possibly arise in traditional coupling systems in that the accessory may cause an involuntary motion by the action of its weight and thus cause an unexpected decoupling of the assembly. Thanks to the system of the present invention, the inertial forces and moments likely to be applied by the probe accessory on the attachment mechanism (the attachment interfaces) of the probe holder do not generally apply in one direction (for example, not along the axis 6) that could cause an unexpected decoupling, particularly related to the weight of the accessory, which advantageously makes it possible to secure the coupling.

It is considered purely for illustrative purposes that the attachment interfaces 14 and 24 are configured so that the rotation OP1 (S4) is performed in the clockwise direction (as illustrated in FIGS. 3 and 6-7). According to one alternative embodiment, the rotation OP1 is performed in the anti-clockwise direction.

According to one example, the second and third attachment interfaces 14 and 24 are configured to be mechanically paired together in a removable manner (or reversibly) by a 1/n turn rotation OP2 of the accessory holder 10 relative to the probe accessory 20, where n is an integer between 2 and 4.

According to one example, n=4. In other words, the second and third attachment interfaces 14 and 24 can be configured to be mechanically paired together in a removable manner by a ¼ turn rotation OP2 of the accessory holder 10 relative to the probe accessory 20 (FIGS. 6-7). This particular configuration is advantageous in that it makes it possible to obtain a good compromise between speed of execution of the mechanical pairing AP2 by a user and robustness of the pairing AP2.

As illustrated in the examples of FIGS. 2-5, the ultrasound probe 2 comprises a transmission output (or transmitter part) 5, located at a distal end of the probe, configured to transmit ultrasound waves W. The accessory holder 10 is configured so that, once the mechanical pairing AP1 has been performed, this transmission output 5 is uncovered or free (that is to say not covered by the accessory holder 10), which makes it possible for ultrasound waves W to be transmitted from this output 5. According to one example, the accessory holder 20 comprises an opening positioned in correspondence with the transmission output 5 in order to uncover the latter when the mechanical pairing AP1 of the accessory holder 10 with the probe 2 is performed.

The design of the attachment interfaces 14 and 24 may be adapted according to the case. Examples of embodiments of these attachment interfaces are now described with reference to FIGS. 9-13.

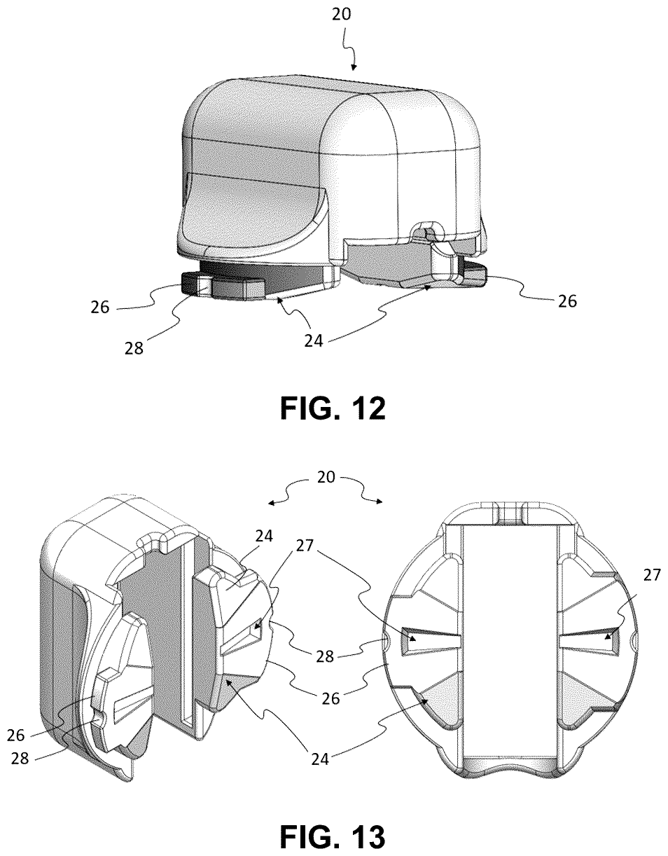

In the following examples, it is assumed that the accessory 20 comprises at least one locking member 26 (FIGS. 12-13) configured to make it possible to lock the attachment interface 24 with the attachment interface 14 during the mechanical pairing AP2. Other alternative embodiments are possible as indicated below.

According to one example illustrated in FIGS. 9-11, the female element, namely the attachment interface 14 in this example, comprises at least one insertion slot 15 and a positioning slide 16 respectively associated with each insertion slot 15. Each insertion slot 15 is configured in order to insert therein a respective locking member 26 of which the accessory 20 is provided. Furthermore, a positioning slide 16 respectively leads to each insertion slot 15 to make it possible to move, by rotating OP2 the probe accessory 20 relative to the accessory holder 10, the respective locking member 26 in the positioning slide 15 up to a locking position P2, this movement thus causing the mechanical pairing AP2 of the accessory holder 10 with the probe accessory 20.

Alternative embodiments according to a reverse configuration are also possible, alternative embodiments wherein the attachment interface 24 of the accessory 20 comprises the insertion slots 16 and a positioning slide 16 respectively associated with each insertion slot 15 and wherein the attachment interface 14 of the accessory holder 10 comprises the corresponding locking member(s) 26. In addition, the various examples described in this invention apply in a similar way to this reverse configuration.

As illustrated in FIGS. 9-13, it is assumed in the following examples purely for illustrative purposes that the accessory 20 comprises two locking members 26. For this purpose, the attachment interface 14 of the accessory holder 10 therefore comprises two insertion slots 15 and two positioning slides 16 respectively leading to these insertion slots 15 to make it possible to move, by rotating OP2 the probe accessory 20 relative to the accessory holder 10, locking members 26 in their respective positioning slide 15 up to a locking position P2, this movement thus causing the mechanical pairing AP2 of the accessory holder 10 with the probe accessory 20.

Nevertheless, it should be noted that alternative embodiments with a different number of locking members 26, and therefore of associated insertion slots 15 and of positioning slides 16, are possible. By way of example, the attachment interface 24 of the accessory 20 may comprise a single locking member 26. In this case, the attachment interface 14 of the accessory holder 10 may comprise a single insertion slot 15 as well as a single associated positioning slide 16 to make it possible to move the single locking member 26 up to the locking position P2. Alternatively, implementations with more than two locking members 26, and as many insertion slots 15 and positioning slides 16, are possible. The use of two or more locking members 26 advantageously makes it possible to make the mechanical pairing AP2 robust while limiting its manufacturing complexity.

Thus, in the example considered (FIGS. 8-13), during the insertion step S2, the insertion operation OP1 brings about the insertion of each of the two locking members 26 into a corresponding insertion slot 15 provided for this purpose in the attachment interface 14 of the accessory holder 10. Once the locking members 26 have been inserted in position into the attachment interface 14, the rotation OP2 of the accessory 20 relative to the accessory holder 10 is carried out about an axis of rotation AX1 from a first angular position P1 into a second angular position P2 (locking position), thus causing the mechanical pairing AP2 of the accessory 20 with the accessory holder 10. The movement of the locking members 26 from the position P1 to the position P2 along their respective positioning slide 16 thus makes it possible to mechanically pair together (AP2) the attachment interfaces 14 and 24. Thus, when the locking members 26 are positioned according to the position P1, the attachment interfaces 12 and 24 are in the first unpaired state ST1. When the locking members 26 are positioned according to the position P2, the attachment interfaces 12 and 24 are in the second paired state ST2.

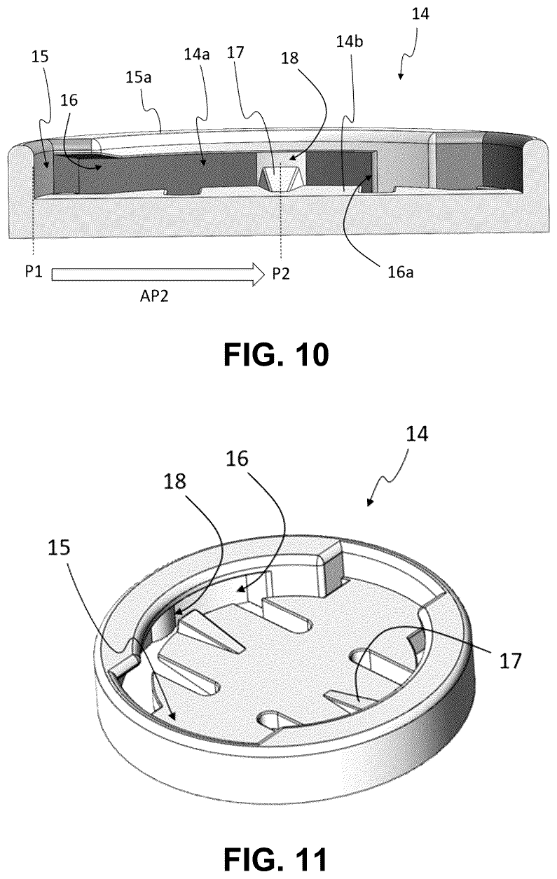

As illustrated particularly in FIGS. 9-11, the attachment interface 14 of the accessory holder 10 may comprise a cavity 14a configured to receive at least one part of the attachment interface 24 of the accessory 20. This cavity 14a facilitates the insertion OP1 of the locking members 26 into their respective insertion slots 15. The positioning slide(s) 16 may then be formed at the edge of this cavity 14a to enable an easy movement of the locking members 26 in the slides 16 by rotating OP2 the accessory 20 relative to the accessory holder 30, about the axis of rotation AX1 (FIGS. 3 and 10).

In the examples considered, the positioning slides 16 are each defined by at least one upper slide edge 15a (FIGS. 9-11). The insertion slots 15 are then each positioned so as to define the entry of a respective positioning slide 15. Each locking member 26 may thus reach a respective slide 16 through the associated insertion slot 15.

The configuration of the locking members 26 (particularly in terms of position, shape, number, etc.) may be adapted depending on that of the insertion slots 15 and of the positioning slides 16, and vice versa. As represented by way of example in FIGS. 12-13, the locking members 26 may be (or comprise) various rigid elements, having for example the form of wings, of protuberances or of pins, capable of being inserted into the insertion slots 15 and of moving by rotating OP2 in the positioning slides 16 up to their respective locking position P2.

As illustrated in FIGS. 9-11, each positioning slide 16 may be provided with a limit stop 16a, marking the locking position P2 of the locking member 26 that it receives. These stops 16a are configured so as to block the locking members 26 when they reach their locking position P2 in their respective positioning slides 16 at the end of rotation OP2. Thanks to these stops 16a, the locking of the accessory 20 at the desired position relative to the accessory holder 10 may be ensured.

According to one example, the second attachment interface 14 comprises two insertion slots 15 (or even 3 or more), potentially of different sizes, these insertion slots being configured so that the respective locking members 26 of the third attachment interface 24 engage therein (or insert therein) during the insertion operation OP1 (Step S2, FIG. 8). As illustrated in FIGS. 9 and 11 for example, one of the insertion slots 15 is larger than the other. Likewise, one of the locking members 26 is larger than the other, each of these locking members 26 being configured to be inserted into an associated insertion slot 15. Advantageously, this configuration makes it possible to form a foolproofing device at the attachment interface 14 to prevent any positioning error of the accessory 20 relative to the accessory holder 10. To this end, one of the two locking members 26 may be configured to be larger (or wider) than another insertion slot 15, namely in this example that designed to receive the other locking member 26. In this way, a single orientation of the accessory 20 is possible relative to the accessory holder 10, which makes it possible to avoid any positioning error at the time of the mechanical pairing AP2. This configuration may be particularly advantageous in some use contexts, for example in a surgical intervention context where the various elements involved should be manipulated effectively, rapidly and reliably, and this despite a restrictive environment (due for example to wearing gloves, sound or other disturbances, stress, high stakes in the event of errors, etc.).

According to one example, the two insertion slots 15 of the attachment interface 14 are positioned diametrically opposite from each other with respect to the axis of rotation AX1 of the accessory 20 relative to the accessory holder 10. This configuration makes a robust and easy mechanical pairing AP2 possible for the user.

According to one example, one of the second and third attachment interfaces 14 and 24 comprises at least one deformable wing 17 configured to deform in response to the movement of a respective locking member 26 in its locking slide 16 up to its locking position P2 so as to cause a frictional force opposing said movement. As illustrated in FIGS. 9-11, it is assumed by way of example that there are two deformable wings 17 in the attachment interface 14 of the accessory holder 10. It should be noted that various configurations particularly in terms of number, positioning and of shape of these deformable wings 17 are possible. Generally, at least one deformable wing 14 may be disposed in the second attachment interface 14, or in the third attachment interface 24, or in both.

More precisely, in the examples illustrated in FIGS. 9-11, the two deformable wings 17 are disposed at the bottom 14b of the cavity 14a arranged in the attachment interface 14 of the accessory holder 10. These wings 17 are configured to deform under the effect of the movement of the locking members 26 from the position P1 to the locking position P2 during the rotation OP2, thus causing a frictional force opposing (contrary to) the movement of the locking members 26. This frictional force results in the rubbing of the deformable wings 17 against a contact surface of the third attachment interface 24 (for example a contact surface of the locking members 26). To this end, the deformable wings 17 may be formed of an elastic material (for example a polyamide (PA) or steel) chosen depending on its elastic properties and needs on a case-by-case basis. The material is preferably chosen with a high elastic limit to guarantee a spring effect of the deformable wings 17.

It is advantageous to place the deformable wing(s) 17 at the attachment interface 14 of the probe holder 10 (rather than at the attachment interface 24) insofar as this makes it possible to obtain a more robust locking system SY3, in particular if it is considered that the probe holder 10 (and more generally the ultrasound probe 2) constitutes a fixed part. Alternatively, the deformable wing(s) 17 may nevertheless be placed at the attachment interface 24 of the accessory 20.

It is advantageous for there to be two or more deformable wings 17, insofar as this makes the locking system SY3 more robust and easier to manipulate. As illustrated, there may be two deformable wings 17, which makes it possible to limit the size and allows a good compromise between the dimensions of the wings and the holding force obtained. Such a compromise may in particular be advantageously reached by positioning two deformable wings 17 diametrically opposite one another relative to the axis of rotation AX1 of the accessory 20 relative to the accessory holder 10.

According to one example illustrated in FIGS. 9-13, the attachment interface 24 comprises two cavities 27 configured for engagement therein by a respective deformable wing 17 when the locking members 26 reach their locking position P2 (at the end of rotation OP2). The arrangement of these cavities 27 facilitates the holding in locking position P2 of the probe accessory 20 relative to the accessory holder 10. To this end, each cavity 27 may have a shape complementary to that of the deformable wings 17 that it receives.

It should be noted that the configuration of these cavities 27, particularly in terms of number and of positioning, may be adapted according to the case depending on the configuration of the deformable wing(s) 27. Generally, at least one of the second and third attachment interfaces 14 and 24 may comprise such a cavity 27 (FIG. 13) configured to receive a corresponding deformable wing 27 when the locking member(s) 26 reach their locking position P2 (at the end of rotation OP2). Alternatively, a single cavity 27 may be arranged in the attachment interface 14 (and/or in the attachment interface 24) in order to insert therein a single deformable wing 27 arranged in the attachment interface 24 (and/or respectively in the attachment interface 14). Alternatively, a plurality of such cavities 27 may be arranged in the attachment interface 14 and/or in the attachment interface 24.

According to one example illustrated in FIGS. 9-13, the second attachment interface 14 comprises two protuberances 18 and the third attachment interface 24 comprises two corresponding cavities (or recesses) 28. Each protuberance 18 is configured to engage in a respective cavity 28 when the locking members 26 reach their locking position P2 at the end of rotation OP2. To this end, the protuberances 18 and the cavities 28 may have complementary shapes in order to mechanically cooperate together according to the male-female type systems. The arrangement of such protuberances 18 and of such cavities 28 advantageously makes it possible to obtain a more robust locking during the mechanical pairing AP2.

Alternative embodiments are nevertheless possible, particularly in terms of number and of positioning of the protuberances 18 and of the cavities 18. Alternatively, the protuberance(s) 18 may be arranged in the attachment interface 24 and the cavity(ies) 28 may be arranged in the attachment interface 14. More generally, one of the second and third attachment interfaces 14 and 24 may thus comprise a cavity 28 and the other comprise a protuberance 18 configured to engage in the cavity 18 when said at least one locking member 26 reaches the locking position P2.

According to one example illustrated in FIGS. 9-13, each locking member 26 of the accessory 20 comprises a respective cavity 28, and reciprocally each positioning slide 16 of the attachment interface 14 comprises a protuberance 18 configured to engage in a respective cavity 28 when the locking members 26 reach their locking position P2 at the end of rotation OP2. Advantageously, this makes it possible to obtain a particularly robust locking during the mechanical pairing AP2.

It should be noted that various configurations of the accessory holder 10 and of the probe accessory 20 are possible, particularly in terms of shape, size, dimensions, etc. The configuration of the accessory holder 10 and of the probe accessory 20 may in particular be adapted depending on the configuration of the ultrasonic probe 2.

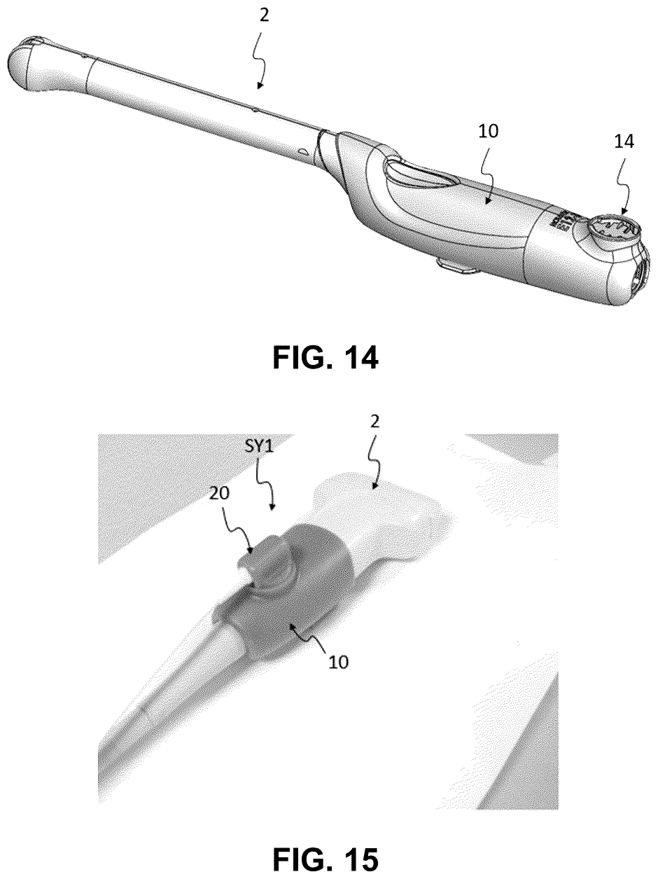

FIGS. 14 and 15 represent two alternative embodiments of the accessory holder 10 and of the probe accessory 20, the respective configurations of which are adapted depending on the configuration of the ultrasound probe 2.

The present invention thus advantageously makes it possible to offer an accessory holder 10 for ultrasound probe ensuring a removable attachment of an accessory 20 to an ultrasound probe 2, this attachment being reliable and robust while guaranteeing good ergonomics of use and easy, effective and fast cleaning.

In particular, the configuration of the accessory holder 10 and of the accessory 20 advantageously makes it possible to mechanically pair by rotating with one another, which facilitates the manipulations by the user (improved ergonomics) and makes it possible to limit the time needed for the pairing and the risks of incorrect positioning.

A user can easily attach and detach the accessory 20 with a good compromise between required force and holding robustness. It is advantageously possible to switch between various accessories 20 that are attached to the ultrasound probe 2 by means of the accessory holder 10.

The particular configuration of the accessory holder 10, and more generally of the accessory system SY1, advantageously allows easy, effective and rapid cleaning of the assembly.

Moreover, the nature of the probe accessory 20 may vary according to the case. The accessory holder 10, and more generally the accessory system SY1, may indeed be adapted to advantageously make it possible to attach various probe accessories 20 to an ultrasound probe 2.

By way of example, the probe accessory 20 comprises at least one of the following:
a sensor;
a gel distributor; and
a light source.

The probe accessory 20 may thus be (or comprise) a sensor, and/or a gel distributor; and/or a light source. Other types of accessories are nevertheless possible.

According to one example, the probe accessory 20 is a subject (a human or an animal for example). In this case, the accessory holder 10 advantageously makes it possible to attach in a removable manner the ultrasound probe 2 to a subject, such as an individual for example. It is for example possible to attach such an ultrasound probe on an anatomical part of a subject in order to perform tests, for example to carry out ultrasound scans.

According to one example, the accessory 20 comprises a sensor, for example a position, motion, pressure, optical, and/or temperature sensor. The accessory 20 comprises for example a position sensor (of the magnetic or other type) configured to detect the position of the ultrasound probe 2 in space when the accessory 20 is attached to the ultrasound probe 2 by means of the accessory holder 10.

According to one example, the accessory 20 comprises a light source (for example a lighting device) configured to illuminate an area of interest close to the ultrasound probe 2 when the accessory 20 is attached to the ultrasound probe 2 by means of the accessory holder 10.

According to one example, the accessory 20 comprises a gel distributor configured to distribute a gel, for example in view of being applied on the ultrasound probe 2 and/or on an area of interest close to the ultrasound probe 2.

According to one example, the probe accessory 20 constitutes a second holder, known as device holder, configured to contain or hold an accessory device, namely for example a sensor and/or a gel distributor and/or a light source such as described above.

According to one example, the accessory 20 is a second holder comprising a housing configured to contain (or receive) a sensor such as described above, for example a position sensor (of the magnetic or other type) and/or a temperature sensor.

Moreover, the probe accessory 20 may have various shape configurations according to the case. According to one example, the accessory 20 forms a rotary knob. Advantageously, this knob thus serves as a gripping element that can be easily manipulated (in particular rotatably) during the mechanical pairing AP2 of the accessory 20 with the accessory holder 10.

As understood by the person skilled in the art, all of the embodiments and alternative embodiments described above, some of which have been deliberately simplified to make them easier to explain, only constitute non-limiting examples of implementation of the present invention. In particular, the person skilled in the art could envisage adapting or combining the embodiments and alternative embodiments described above in order to address a specific need.

The present invention is therefore not limited to the examples of embodiments described above but particularly extends to a control method that would include secondary steps without departing from the scope of the present invention. The same would apply to a control device, or more generally a control system, for implementing such a method.

1. Accessory holder for ultrasound probe, the accessory holder comprising:
a first attachment interface configured to mechanically pair with an ultrasound probe; and
a second attachment interface, of shape complementary to a third attachment interface with which a probe accessory is provided, configured to mechanically pair in a removable manner with said third attachment interface by rotating the probe accessory relative to the accessory holder.

2. Accessory holder according to claim 1, wherein one of the second and third attachment interfaces constitutes a male element and the other constitutes a female element, said male and female elements being configured to rotatably mechanically pair by inserting the male element into the female element.

3. Accessory holder according to claim 2, wherein the second attachment interface of the accessory holder constitutes the female element.

4. Accessory holder according to claim 2, wherein the second attachment interface is configured to mechanically pair in a removable manner with said third attachment interface by mechanical pairing successively comprising inserting the male element into the female element in a direction of insertion and rotating the probe accessory relative to the accessory holder about an axis of rotation corresponding to the direction of insertion.

5. Accessory holder according to claim 2, wherein the female element comprises:
at least one insertion slot configured to insert therein a respective locking member with which the male element is provided; and
a positioning slide respectively leading to each insertion slot to make it possible to move, by rotating the probe accessory relative to the accessory holder, the respective locking member in the positioning slide up to a locking position thus causing about the mechanical pairing of the accessory holder with the probe accessory.

6. Accessory holder according to claim 5, wherein one of the second and third attachment interfaces comprises at least one deformable wing configured to deform in response to the movement of the respective locking member in the locking slide up to the locking position so as to cause a frictional force opposing said movement.

7. Accessory holder according to claim 6, wherein said at least one deformable wing is comprised in the second attachment interface of the accessory holder.

8. Accessory holder according to claim 7, wherein the second attachment interface comprises two deformable wings.

9. Accessory holder according to claim 6, wherein one of the second and third attachment interfaces comprises at least one first cavity, said at least one deformable wing and said at least one first cavity being configured so that each deformable wing engages in a first cavity when said at least one locking member reaches the locking position.

10. Accessory holder according to claim 5, wherein the second attachment interface comprises at least two insertion slots of different size configured for engagement therein by respective locking members of the third attachment interface.

11. Accessory holder according to claim 5, wherein one of the second and third attachment interfaces comprises a second cavity and the other comprises a protuberance configured to engage in the second cavity when said at least one locking member reaches the locking position.

12. Accessory holder according to claim 1, wherein the second and third attachment interfaces are configured to be mechanically paired in a removable manner by a ¼ turn rotation of the accessory holder relative to the probe accessory.

13. Accessory system for ultrasound probe comprising:
an accessory holder such as defined in claim 1, and
a probe accessory comprising a third attachment interface of shape complementary to the second attachment interface of the accessory holder, wherein the second and third attachment interfaces are configured to mechanically pair in a removable manner by rotating the probe accessory relative to the accessory holder.

14. System according to claim 13, wherein one of the second and third attachment interfaces constitutes a male element and the other constitutes a female element, said male and female elements being configured to rotatably mechanically pair by inserting the male element into the female element,
wherein the second and third attachment interfaces are configured to mechanically pair in a removable manner by mechanical pairing successively comprising inserting the male element into the female element in a direction of insertion and rotating the probe accessory relative to the accessory holder about an axis of rotation corresponding to the direction of insertion.

15. System according to claim 13, wherein the probe accessory comprises at least one of the following:
a sensor,
a gel distributor, and
a light source.

16. System according to claim 15, wherein the probe accessory comprises a position sensor.

17. System according to claim 13, wherein the probe accessory forms a rotary knob.
